(19) United States
(12) Patent Application Publication
(10) Pub. No.: US 2015/0359505 A1
(43) Pub. Date: Dec. 17, 2015

(54) RADIATION IMAGING APPARATUS, METHOD FOR CONTROLLING THE SAME, AND STORAGE MEDIUM

(71) Applicant: CANON KABUSHIKI KAISHA, Tokyo (JP)

(72) Inventor: Mina Hoshino, Fujisawa-shi (JP)

(21) Appl. No.: 14/716,691

(22) Filed: May 19, 2015

(30) Foreign Application Priority Data
Jun. 11, 2014 (JP) 2014-120771

(51) Int. Cl. A61B 6/00; G01T 1/20

(52) U.S. Cl. CPC A61B 6/586; G01T 1/2018; A61B 6/42; A61B 6/54

(57) ABSTRACT

A radiation imaging apparatus for capturing a radiation image based on radiation which penetrates a subject includes a detection unit and a position determination unit. The detection unit detects a physical quantity which is changed when the radiation imaging apparatus takes an impact. The position determination unit determines an impact position subjected to the impact, based on the physical quantity detected by the detection unit.

### BACKGROUND OF THE INVENTION

1. Field of the Invention

The present invention relates to a radiation imaging apparatus for capturing a radiation image based on radiation which has penetrated a subject, a method for controlling the radiation imaging apparatus, a radiation imaging system including the radiation imaging apparatus, and a program for causing a computer to execute the control method.

2. Description of the Related Art

Radiation images are used in medical diagnosis. A radiation image is obtained by irradiating a patient as a subject with radiation and capturing the radiation which has penetrated the patient. A Flat Panel Detector (FPD), which is widely used in recent years, detects radiation which has penetrated a subject and then converts the radiation into electric energy to obtain a radiation image. Since the FPD is a precision apparatus, it is not robust against an impact although anti-impact measures have been taken. However, a commonly used FPD lacks a method for analyzing information of an impact when the FPD takes the impact, i.e., there is no way to know what kind of impact has been exerted on the FPD.

Japanese Patent Application Laid-Open No. 2011-67334 discusses a technique for preventing a failed FPD from capturing an image. The technique determines whether the value of acceleration detected by a 3-axis acceleration sensor provided within the FPD exceeds a threshold value, and, if the acceleration value exceeds the threshold value, determines that the FPD has been impacted in a serious manner. Further, the technique automatically performs self-diagnosis by using a second threshold value to determine whether the FPD is usable.

Further, Japanese Patent Application Laid-Open No. 2012-45243 discusses a technique for enabling remote maintenance. The technique detects vibration, pressure, temperature, and other disturbances by using a sensor provided in an image capturing unit, performs self-diagnosis at short intervals, and then transmits a diagnostic result to a server to notify a maintenance contractor of a current condition.

However, the techniques discussed in Japanese Patent Application Laid-Open No. 2011-67334 and Japanese Patent Application Laid-Open No. 2012-45243 have an issue that, it is difficult to grasp what kind of impact has been exerted on the image capturing unit (imaging apparatus) when the unit takes the impact. Therefore, if the image capturing unit (imaging apparatus) has failed due to the impact, a much amount of labor and time needs to be spent to analyze which portion of the image capturing unit (imaging apparatus) is damaged, resulting in a prolonged downtime of a user.

### SUMMARY OF THE INVENTION

The present invention is directed to providing a mechanism for grasping what kind of impact has been exerted on an imaging apparatus when the imaging apparatus has taken the impact.

According to an aspect of the present invention, a radiation imaging apparatus for capturing a radiation image based on radiation which penetrates a subject, the radiation imaging apparatus includes a detection unit configured to detect a physical quantity which is changed when the radiation imaging apparatus takes an impact, and a position determination unit configured to determine an impact position subjected to the impact, based on the physical quantity detected by the detection unit.

Further features of the present invention will become apparent from the following description of exemplary embodiments with reference to the attached drawings.

### BRIEF DESCRIPTION OF THE DRAWINGS

FIG. 1 schematically illustrates a configuration of a radiation imaging system according to a first exemplary embodiment of the present invention.

FIGS. 2A and 2B illustrate examples of appearances of a radiation imaging apparatus according to the first exemplary embodiment of the present invention.

FIG. 3 schematically illustrates a configuration of the radiation imaging apparatus according to the first exemplary embodiment of the present invention.

FIGS. 4A and 4B illustrate features of the radiation imaging apparatus according to the first exemplary embodiment of the present invention.

FIG. 5 illustrates an example of an internal configuration of an overall control circuit unit illustrated in FIG. 3.

FIGS. 6A, 6B, and 6C illustrate specific examples of determination processing by a processing unit illustrated in FIG. 5.

FIG. 7 illustrates an example of an internal configuration of the radiation imaging apparatus within a housing according to the first exemplary embodiment of the present invention.

FIG. 8 illustrates a specific example of determination processing by an impact position determination unit illustrated in FIG. 5.

FIG. 9 is a schematic view illustrating the housing of the radiation imaging apparatus illustrated in FIG. 7 viewed from a side surface.

FIGS. 10A to 10D illustrate examples of control processing by a control unit illustrated in FIG. 5.

### DESCRIPTION OF THE EMBODIMENTS

A first exemplary embodiment of the present invention will be described below. The present invention provides workings for figuring-out a kind of impact which is applied to an imaging apparatus when it takes the impact. A radiation imaging apparatus for capturing a radiation image based on radiation which penetrates a subject includes an impact detection unit configured to detect a physical quantity which is changed when the radiation imaging apparatus takes the impact, and an impact position determination unit configured to determine an impact position which has been impacted, based on the physical quantity detected by the impact detection unit.

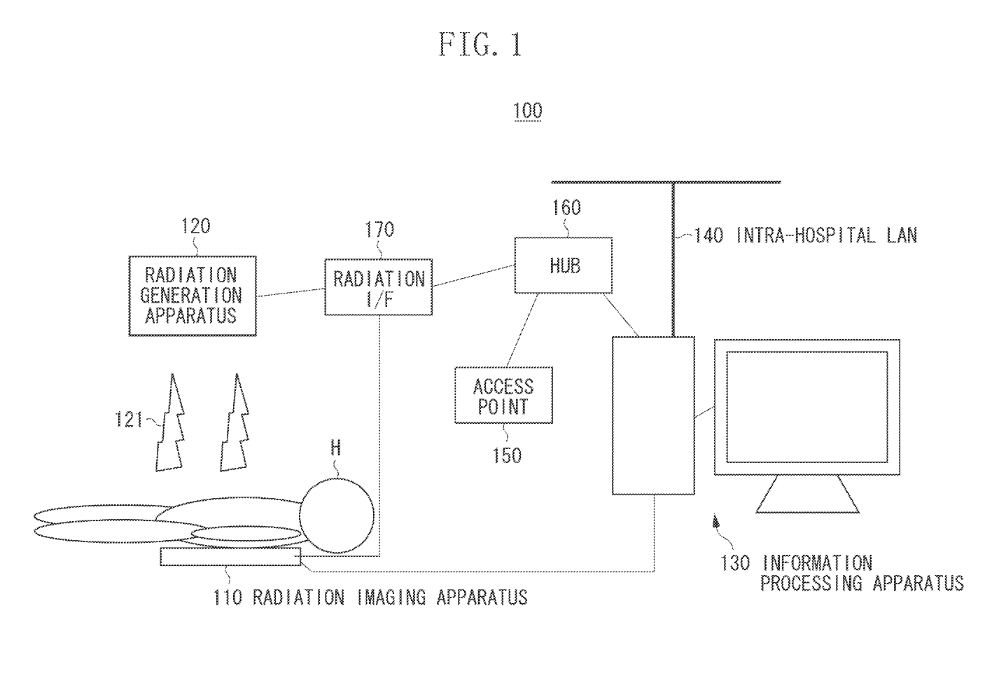

FIG. 1 schematically illustrates a configuration of a radiation imaging system 100 according to the first exemplary embodiment of the present invention.

As illustrated in FIG. 1, the radiation imaging system 100 includes a radiation imaging apparatus 110, a radiation generation apparatus 120, an information processing apparatus 130, an intra-hospital local area network (LAN) 140, an access point 150, a hub 160, and a radiation interface 170.

The radiation imaging apparatus 110 captures radiation images based on radiation 121 which penetrates a subject H. The radiation imaging apparatus 110 includes, for example, a portable type cassette flat panel detector.

The radiation generation apparatus 120 includes, for example, a radiation tube and a rotor for accelerating electrons with a high voltage to cause them strike the anode, thus generating the radiation 121 such as X-ray. Although the present specification describes X-ray as an example of the radiation according to the present invention, the radiation in the present invention is not limited to X-ray, and includes alpha ray, beta ray, and gamma ray.

The information processing apparatus 130 displays a radiation image captured by the radiation imaging apparatus 110 on a display unit, and issues an instruction with respect to an imaging mode which is input via an operation input unit.

The intra-hospital LAN 140 is a local area network built in a hospital.

The access point 150 is a radio wave relay unit for connecting between terminals.

The HUB 160 is an apparatus for connecting a plurality of networking apparatuses.

The radiation interface (radiation I/F) 170 includes a circuit for mediating communication to monitor the statuses of the radiation imaging apparatus 110 and the radiation generation apparatus 120. For example, the radiation I/F 170 controls, for example, emission of the radiation 121 from the radiation generation apparatus 120, and imaging of the subject H by the radiation imaging apparatus 110.

In the radiation imaging system 100 illustrated in FIG. 1, the subject H (a patient) is irradiated with the radiation 121 emitted from the radiation generation apparatus 120. The radiation imaging apparatus 110 detects the radiation 121 which has penetrated the subject H to generate a radiation image.

In the “automatic detection mode” which has been widely used in recent years, the radiation generation apparatus 120 not having the radiation I/F 170 emits the radiation 121, and the radiation imaging apparatus 110 automatically accumulates image signals (electric charges) to generate a radiation image. Such a radiation imaging system not having the radiation I/F 170 and operating in the automatic detection mode is also applicable to the present exemplary embodiment.

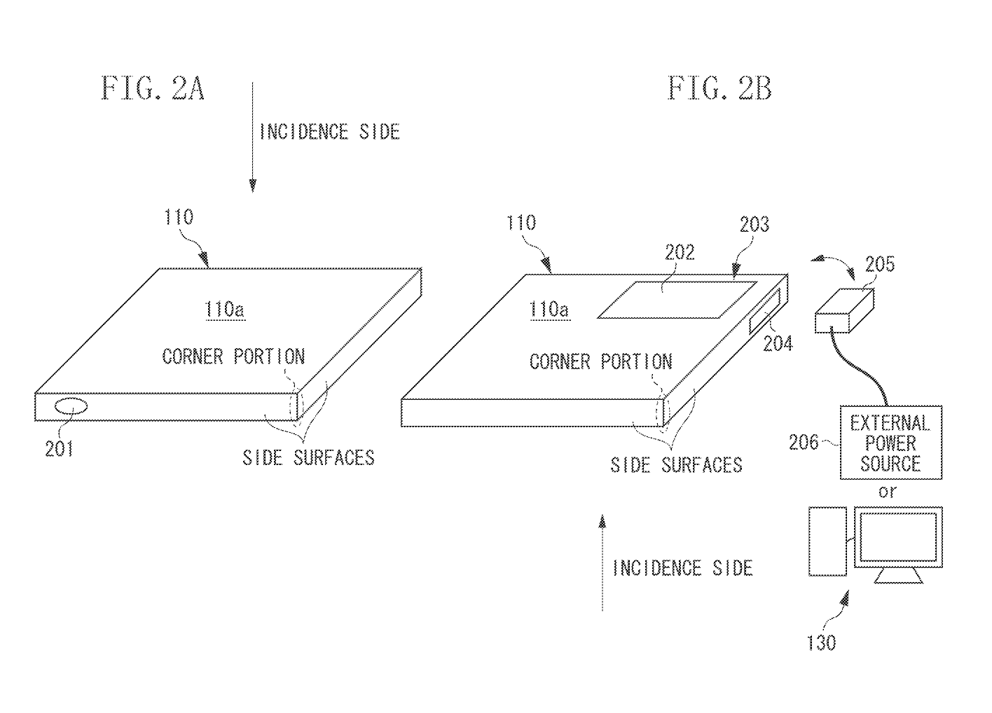

FIGS. 2A and 2B illustrate examples of appearances of the radiation imaging apparatus 110 according to the first exemplary embodiment of the present invention. FIGS. 2A and 2B illustrate a portable type radiation imaging apparatus 110. FIG. 2A illustrates a housing 110a of the radiation imaging apparatus 110 where the radiation is incident from an upper side. FIG. 2B illustrates the housing 110a of the radiation imaging apparatus 110 where the radiation is incident from a lower side.

As illustrated in FIGS. 2A and 2B, a power button 201, a battery unit 202, an attachment portion 203, a connector contact portion 204, a sensor cable 205, and an external power source 206 are provided on the housing 110a of the radiation imaging apparatus 110. In the example illustrated in FIGS. 2A and 2B, the housing 110a of the radiation imaging apparatus 110 has an approximate rectangular parallelepiped shape, and is formed of four side surfaces and four corner portions each being formed of two adjacent side surfaces out of the four side surfaces. In the present exemplary embodiment, the housing 110a of the radiation imaging apparatus 110 is not limited to an approximate rectangular parallelepiped shape illustrated in FIGS. 2A and 2B, and may be formed in various shapes. For example, in the present exemplary embodiment, it is suitable that the housing 110a of the radiation imaging apparatus 110 has at least four side surfaces and a plurality of corner portions each being formed of two adjacent side surfaces out of the four side surfaces.

As illustrated in FIG. 2A, when the radiation is incident from the upper side, the power button 201 is disposed on a side surface of the housing 110a of the radiation imaging apparatus 110.

As illustrated in FIG. 2B, when the radiation is incident from the lower side, the attachment portion 203 for attaching the battery unit 202 is disposed on the rear surface of the housing 110a of the radiation imaging apparatus 110. The battery unit 202 is charged by a dedicated battery charger. The battery unit 202 can be attached by inserting its convex portion into the attachment portion 203 of the radiation imaging apparatus 110.

As illustrated in FIG. 2B, when the sensor cable 205 for enabling external connection is attached to the connector contact portion 204 disposed on a side surface of the housing 110a, power can be supplied from the external power source 206 to the radiation imaging apparatus 110. Further, when the sensor cable 205 for enabling external connection is connected to the connector contact portion 204, wired connection to the information processing apparatus 130 is enabled. In this case, the connector contact portion 204 and the sensor cable 205 can be connected with each other by, for example, providing an adsorption plate on the connector contact portion 204 and a magnet on the sensor cable 205.

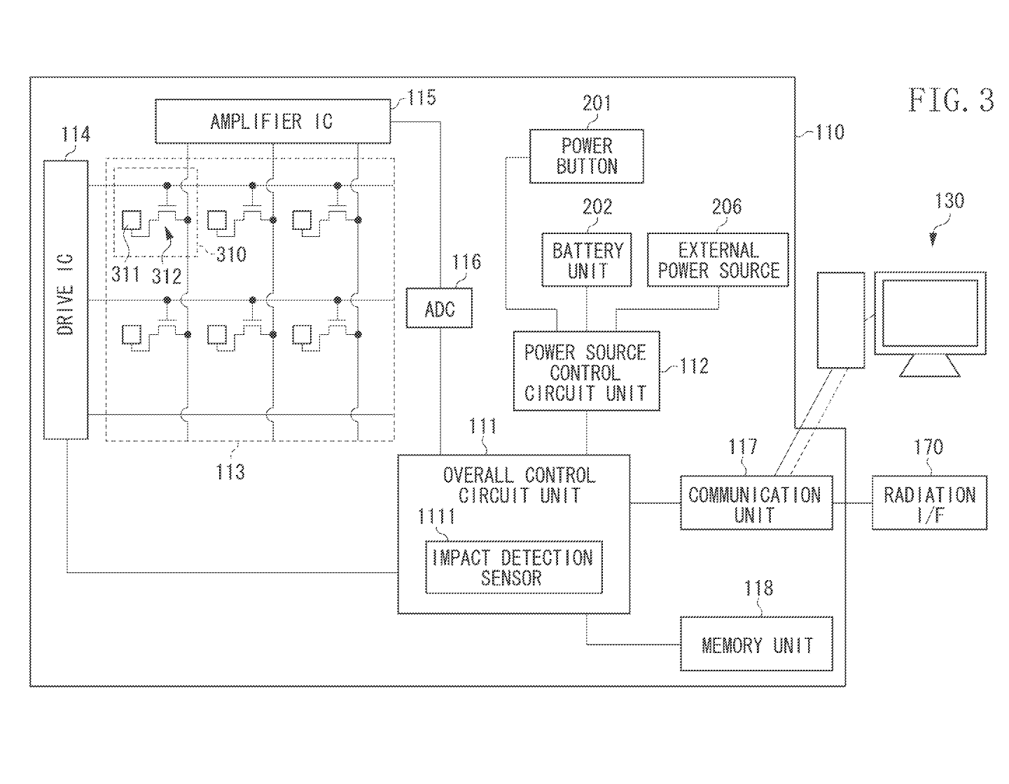

FIG. 3 schematically illustrates a configuration of the radiation imaging apparatus 110 according to the first exemplary embodiment of the present invention.

As illustrated in FIG. 3, the radiation imaging apparatus 110 includes as its components an overall control circuit unit 111, a power source control circuit unit 112, a radiation detection unit 113, a drive integrated circuit (IC) 114, an amplifier IC 115, an analog-to-digital converter (ADC) 116, a communication unit 117, a memory unit 118, a power button 201, a battery unit 202, and an external power source 206. More specifically, the housing 110a of the radiation imaging apparatus 110 illustrated in FIGS. 2A and 2B houses the overall control circuit unit 111, the power source control circuit unit 112, the radiation detection unit 113, the drive IC 114, the amplifier IC 115, the ADC 116, the communication unit 117, the memory unit 118, and the battery unit 202. Referring to FIG. 3, components identical to those illustrated in FIGS. 2A and 2B are assigned the same reference numeral, and redundant descriptions will be omitted.

The overall control circuit unit 111 controls overall operations of the radiation imaging apparatus 110. For example, an impact detection sensor 1111 is provided in the overall control circuit unit 111.

Depending on the operating state of the power button 201 and the connections with the battery unit 202 and the external power source 206, the power source control circuit unit 112 controls power supply from respective power sources to each construction unit and monitors the remaining battery capacity. For example, the battery unit 202 supplies power to the radiation detection unit 113. For example, the power source control circuit unit 112 transforms power from the battery unit 202 into a predetermined voltage and supplies it to each construction unit.

The radiation detection unit 113 detects the radiation 121 which has penetrated the subject H as an image signal (electric charge). More specifically, the radiation detection unit 113 includes a group of pixels 310 arranged in 2-dimensional array form, and phosphor (not illustrated) disposed on the pixel group between the relevant pixel group and the subject H. A pixel 310 includes a photoelectric conversion element 311 and a switch element 312. The switch element 312 includes, for example, a thin-film transistor (TFT) formed of amorphous silicon on a glass substrate.

The phosphor absorbs the radiation 121 which penetrates the subject H and then converts energy into light (i.e., converts the radiation 121 into light). The photoelectric conversion element 311 converts the light generated through conversion by the phosphor into image signals (electric charges) which are electrical signals to be accumulated. Thus, the radiation detection unit 113 configures a radiation imaging unit for detecting the radiation 121 which has penetrated the subject H to acquire image signals. The drive IC 114 configures a drive circuit unit for applying a drive signal to the radiation detection unit 113. More specifically, when the pixels 310 in a certain row are selected by the drive IC 114 via the drive signal, the switch elements 312 of the pixels 310 in the relevant certain row sequentially is turned ON, and image signals (electric charges) accumulated in the photoelectric conversion elements 311 of the pixels 310 in the relevant certain row are output to signal wires connected to respective pixels 310. The amplifier IC 115 sequentially reads the image signals output to the signal wires. The ADC 116 converts an analog image signal read by the amplifier IC 115 into a digital image signal and then outputs the digital image signal to the overall control circuit unit 111 as radiation image data. In other words, the ADC 116 configures an analog-to-digital (A/D) conversion unit for converting an analog image signal read by the amplifier IC 115 into digital data. The overall control circuit unit 111 temporarily stores in the memory unit 118 the radiation image data output from the ADC 116. The memory unit 118 configures a storage unit for storing radiation image data based on the image signal acquired by the radiation detection unit 113.

The communication unit 117, for example, wirelessly communicates with the information processing apparatus 130 and the radiation I/F 170. In this case, the communication unit 117 configures a wireless communication unit for wirelessly communicating with an external apparatus. The communication unit 117 may communicate by cable with the information processing apparatus 130 and the radiation I/F 170 via the connector contact portion 204 and the sensor cable 205.

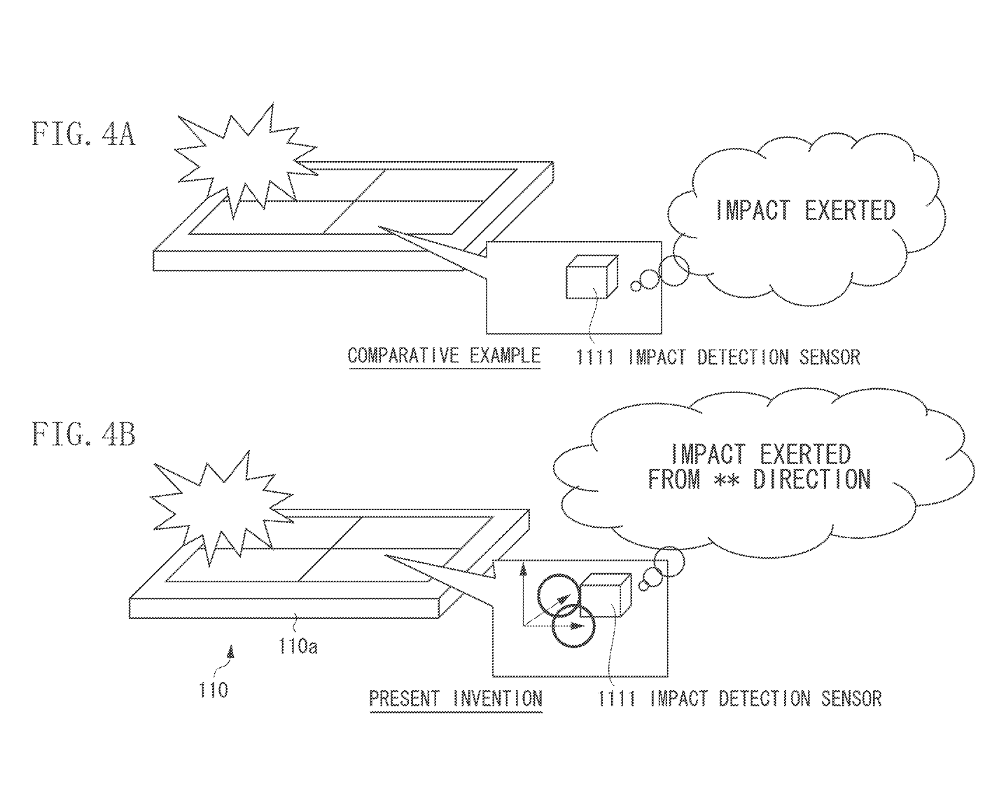

FIGS. 4A and 4B illustrate a feature of the radiation imaging apparatus 110 according to the first exemplary embodiment of the present invention.

FIG. 4A illustrates a feature of a radiation imaging apparatus according to a comparative example. More specifically, FIG. 4A illustrates how the apparatus determines whether it takes the impact, based on information detected by an impact detection sensor.

FIG. 4B illustrates a feature of the radiation imaging apparatus 110 according to the present exemplary embodiment. More specifically, FIG. 4B illustrates how the apparatus determines whether the apparatus has taken an impact, based on information detected by the impact detection sensor 1111. If the apparatus has taken the impact, it determines the position subjected to the impact. The impact detection sensor 1111 according to the present exemplary embodiment detects a physical quantity which changes when the radiation imaging apparatus 110 takes the impact. The radiation imaging apparatus 110 according to the present exemplary embodiment acquires the physical quantity in a plurality of axis directions and then performs analysis to determine the position subjected to the impact. The impact detection sensor 1111 is, for example, a 3-axis acceleration sensor which is capable of detecting the acceleration in the 3-axis directions (in the X-axis, the Y-axis, and the Z-axis directions). Alternatively, the impact detection sensor 1111 may employ three 1-axis acceleration sensors or two 2-axis acceleration sensors in different axis directions.

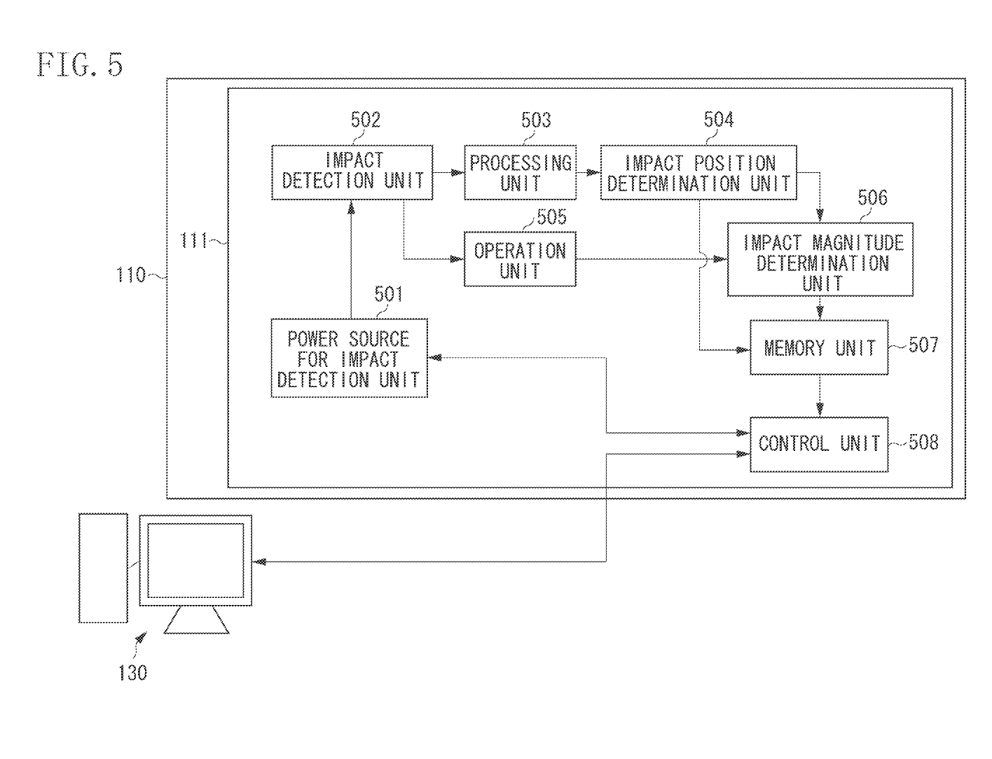

FIG. 5 illustrates an example of an internal configuration of the overall control circuit unit 111 illustrated in FIG. 3.

The overall control circuit unit 111 in the radiation imaging apparatus 110 includes as its components a power source for impact detection unit 501, an impact detection unit 502, a processing unit 503, an impact position determination unit 504, an calculation unit 505, an impact magnitude determination unit 506, a memory unit 507, and a control unit 508.

The power source for impact detection unit 501 constantly supplies power to the impact detection unit 502 to constantly carry out impact detection. In other words, the power source for impact detection unit 501 is a power supply unit for supplying power to the impact detection unit 502 even when power (the battery unit 202 and the external power source 206) is not input to the radiation imaging apparatus 110.

The impact detection unit 502 includes as its components the impact detection sensor 1111 illustrated in FIGS. 3 and 4 to detect the physical quantity which changes when the radiation imaging apparatus 110 takes the impact. More specifically, in the present exemplary embodiment, the impact detection unit 502 detects the physical quantity in a plurality of axis directions (for example, in the X-axis, the Y-axis, and the Z-axis directions) in the radiation imaging apparatus 110. Further, the impact detection unit 502 detects the impact when the magnitude of the detected physical quantity is equal to or greater than a predetermined threshold value.

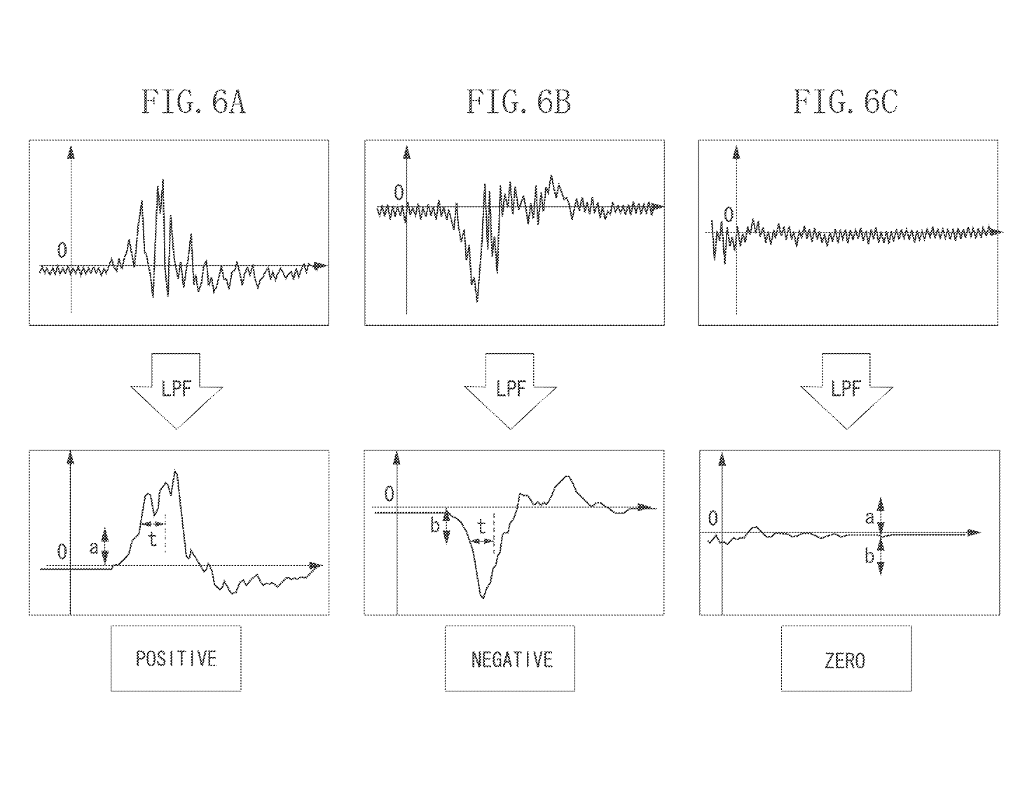

The processing unit 503 performs determination processing for determining in each axis direction the output waveform type (which indicates change of the physical quantity) related to the physical quantity in a plurality of axis directions detected by the impact detection unit 502. More specifically, the processing unit 503 performs determination processing for determining whether the output waveform type is a first output waveform, a second output waveform, or a third output waveform. The first output waveform has a time period equal to or longer than a predetermined period, during which the magnitude is equal to or greater than a first threshold value which is a positive threshold value. The second output waveform has a time period equal to or longer than a predetermined period, during which the magnitude is equal to or smaller than a second threshold value which is a negative threshold value. The third output waveform is other than the first output waveform and the second output waveform. The processing unit 503 performs the above-described determination processing when the impact is detected by the impact detection unit 502. Specific examples of the determination by the processing unit 503 will be described below with reference to FIGS. 6A to 6C.

FIGS. 6A to 6C illustrate specific examples of the determination made by the processing unit 503 illustrated in FIG. 5.

The examples illustrated in FIGS. 6A to 6C indicate a case where an acceleration sensor is used as the impact detection sensor 1111, i.e., a case where acceleration is adopted as the physical quantity in a certain axis direction detected by the impact detection unit 502.

Referring to FIGS. 6A to 6C, waveforms at the top indicate output waveforms of acceleration in a certain axis direction detected by the impact detection unit 502.

In the case of the output waveform illustrated in FIG. 6A, the processing unit 503 first performs low-pass filter (hereinafter referred to as “LPF”) processing on the output waveform and then performs determination processing for determining the output waveform as a first output waveform having a time period equal to or longer than a predetermined period (a time period t), during which the magnitude is equal to or greater than a first threshold value (a threshold value a) which is a positive threshold value. In other words, in the case of the output waveform illustrated in FIG. 6A, the processing unit 503 performs determination processing for determining the output waveform as a “Positive” type output waveform which is a first output waveform.

In the case of the output waveform illustrated in FIG. 6B, the processing unit 503 first performs low-pass filter (hereinafter referred to as “LPF”) processing on the output waveform and then performs determination processing for determining the output waveform as a second output waveform having a time period equal to or longer than a predetermined period (a time period t), during which the magnitude is equal to or smaller than a second threshold value (a threshold value b) which is a negative threshold value. In other words, in the case of the output waveform illustrated in FIG. 6B, the processing unit 503 performs determination processing for determining the output waveform as a “Negative” type output waveform which is a second output waveform.

In the case of the output waveform illustrated in FIG. 6C, the processing unit 503 first performs LPF processing on the output waveform and then performs determination processing for determining the output waveform as a third output waveform which is neither the first nor the second output waveform. In other words, in the case of the output waveform illustrated in FIG. 6C, the processing unit 503 performs determination processing for determining the output waveform as a “Zero” type output waveform which is a third output waveform.

Descriptions of FIG. 5 will be resumed below.

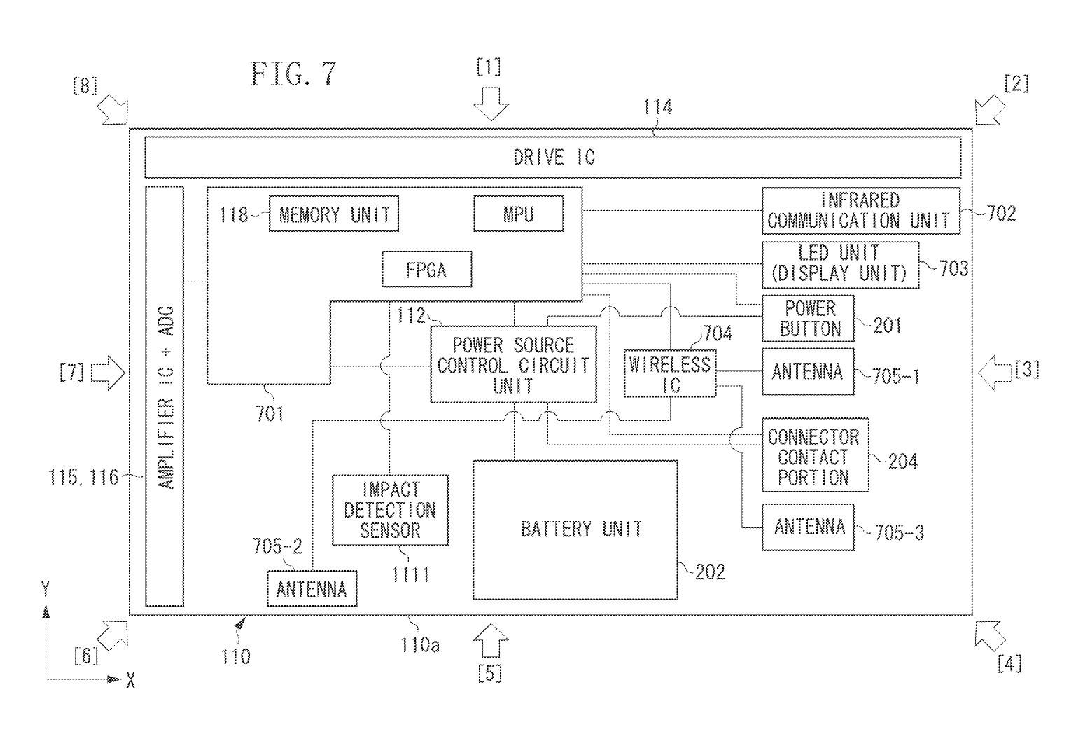

Based on the physical quantity detected by the impact detection unit 502, the impact position determination unit 504 determines the impact position, i.e., a position on the housing 110a of the radiation imaging apparatus 110 subjected to the impact from external. More specifically, the impact position determination unit 504 determines the impact position on the radiation imaging apparatus 110 according to the output waveform type in each axis direction obtained as a result of the determination processing by the processing unit 503. A specific example of the determination processing by the impact position determination unit 504 will be described below with reference to FIG. 7.

FIG. 7 illustrates an example of an internal configuration of the housing 110a of the radiation imaging apparatus 110 according to the first exemplary embodiment of the present invention. More specifically, FIG. 7 illustrates an example of an arrangement of an electric circuit unit viewed from the side opposite to the incidence side of radiation. Referring to FIG. 7, the horizontal direction is the X-axis direction, and the vertical direction is the Y-axis direction. Referring to FIG. 7, components identical to those illustrated in FIGS. 2 and 3 are assigned the same reference numeral, and redundant descriptions will be omitted.

A comprehensive control circuit unit 701 integrally controls operations of the radiation imaging apparatus 110, and includes as its components a micro processing unit (MPU), a Field-Programmable Gate Array (FPGA), and a memory unit 118. The comprehensive control circuit unit 701 is configured such that the impact detection sensor 1111 is removed from the overall control circuit unit 111 illustrated in FIG. 3 and the memory unit 118 is added to the overall control circuit unit 111. Referring to FIG. 7, the impact detection sensor 1111 is connected to the comprehensive control circuit unit 701.

An infrared communication unit 702 and a light emitting diode (LED) unit (display unit) 703 are connected to the comprehensive control circuit unit 701.

A wireless IC 704 and antennas 705-1 to 705-3 configure the communication unit 117 illustrated in FIG. 3.

The comprehensive control circuit unit 701 and the power source control circuit unit 112 are connected to the connector contact portion 204.

As described with reference to FIGS. 2A and 2B, the housing 110a of the radiation imaging apparatus 110 illustrated in FIG. 7 has an approximate rectangular parallelepiped shape, and is formed of four side surfaces and four corner portions each being formed of two adjacent side surfaces out of the four side surfaces.

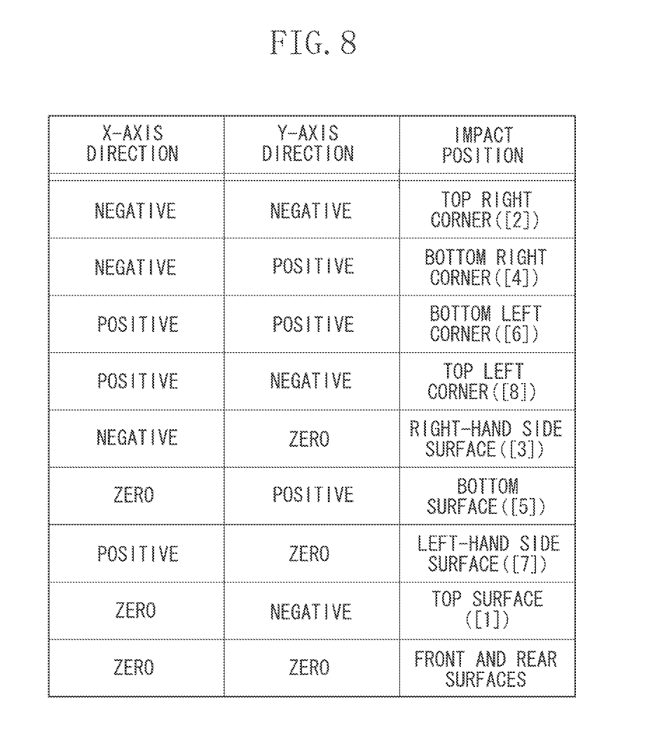

FIG. 8 illustrates a specific example of determination processing by the impact position determination unit 504 illustrated in FIG. 5. FIG. 8 is a table illustrating impact positions corresponding to combinations of output waveform types in respective axis directions. This table is stored, for example, in the impact position determination unit 504.

The impact position determination unit 504 acquires the output waveform type in each axis direction obtained as a result of the determination processing by the processing unit 503 and then determines the impact position on the radiation imaging apparatus 110 referring to the table illustrated in FIG. 8.

More specifically, when the output waveform in the X-axis direction acquired from the processing unit 503 is a “Negative” type output waveform and the output waveform in the Y-axis direction acquired therefrom is a “Negative” type output waveform, the impact position determination unit 504 determines that the position of the top right corner [2] illustrated in FIG. 7 is the impact position.

When the output waveform in the X-axis direction acquired from the processing unit 503 is a “Negative” type output waveform and the output waveform in the Y-axis direction acquired therefrom is a “Positive” type output waveform, the impact position determination unit 504 determines that the position of the bottom right corner [4] illustrated in FIG. 7 is the impact position.

When the output waveform in the X-axis direction acquired from the processing unit 503 is a “Positive” type output waveform and the output waveform in the Y-axis direction acquired therefrom is a “Positive” type output waveform, the impact position determination unit 504 determines that the position of the bottom left corner [6] illustrated in FIG. 7 is the impact position.

When the output waveform in the X-axis direction acquired from the processing unit 503 is a “Positive” type output waveform and the output waveform in the Y-axis direction acquired therefrom is a “Negative” type output waveform, the impact position determination unit 504 determines that the position of the top left corner [8] illustrated in FIG. 7 is the impact position.

In this way, the impact position determination unit 504 determines at least a corner portion which has been subjected to an impact, among a plurality of the corner portions of the housing 110a.

When the output waveform in the X-axis direction acquired from the processing unit 503 is a “Negative” type output waveform and the output waveform in the Y-axis direction acquired therefrom is a “Zero” type output waveform, the impact position determination unit 504 determines that the position of the right-hand side surface [3] illustrated in FIG. 7 is the impact position.

When the output waveform in the X-axis direction acquired from the processing unit 503 is a “Zero” type output waveform and the output waveform in the Y-axis direction acquired therefrom is a “Positive” type output waveform, the impact position determination unit 504 determines that the position of the bottom surface [5] illustrated in FIG. 7 is the impact position.

When the output waveform in the X-axis direction acquired from the processing unit 503 is a “Positive” type output waveform and the output waveform in the Y-axis direction acquired therefrom is a “Zero” type output waveform, the impact position determination unit 504 determines that the position of the left-hand side surface [7] illustrated in FIG. 7 is the impact position.

When the output waveform in the X-axis direction acquired from the processing unit 503 is a “Zero” type output waveform and the output waveform in the Y-axis direction acquired therefrom is a “Negative” type output waveform, the impact position determination unit 504 determines that the position of the top surface [1] illustrated in FIG. 7 is the impact position.

When the output waveform in the X-axis direction acquired from the processing unit 503 is a “Zero” type output waveform and the output waveform in the Y-axis direction acquired therefrom is a “Zero” type output waveform, the impact position determination unit 504 determines that either of the front or rear surface of the radiation imaging apparatus 110 is the impact position.

In the present exemplary embodiment, the impact position determination unit 504 determines the impact position on the radiation imaging apparatus 110, as illustrated in FIGS. 7 and 8. Although, in FIGS. 7 and 8, the impact position on the radiation imaging apparatus 110 is determined based on the output waveform types only in the X-axis and the Y-axis directions, the present invention is not limited thereto. For example, the present invention includes a configuration in which a table as illustrated in FIG. 8 is prepared in advance in consideration of the Z-axis direction which is perpendicular to the paper surface illustrated in FIG. 7, and the impact position on the radiation imaging apparatus 110 is determined by referring to the table.

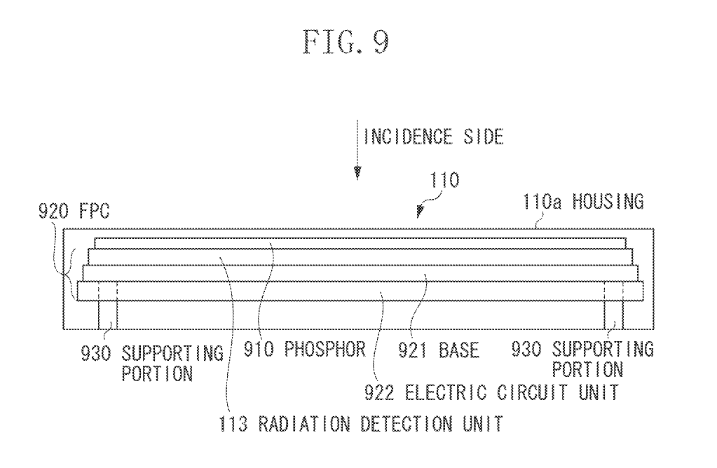

FIG. 9 is a schematic view illustrating the housing 110a of the radiation imaging apparatus 110 illustrated in FIG. 7 viewed from a side surface. FIG. 9 illustrates a case where the radiation is incident from an upper side.

The housing 110a of the radiation imaging apparatus 110 includes a phosphor 910, a Flexible Printed Circuit Board (FPC) 920 (including the radiation detection unit 113 illustrated in FIG. 3, a base 921, and an electric circuit unit 922), and supporting portions 930.

The phosphor 910 converts incident radiation into light. The electric circuit unit 922 is equivalent to the electric circuit unit illustrated in FIG. 7, and is supported by the base 921. The supporting portions 930 support the base 921 at a plurality of positions. Although the FPC 920 actually exists on both of the side surface of the drive IC 114, and the side surface of the amplifier IC 115 and the ADC 116 illustrated in FIG. 7, only one FPC 920 is illustrated in FIG. 9.

Descriptions of FIG. 5 will be resumed below.

The calculation unit 505 calculates the impact value based on the physical quantity detected by the impact detection unit 502. More specifically, the calculation unit 505 calculates the impact value based on the sum of squares of the physical quantities in a plurality of axis directions (for example, in the X-axis, the Y-axis, and the Z-axis directions). For example, the calculation unit 505 calculates the impact value by using the following formula.

Impact value=(Acceleration in X-axis direction)²+(Acceleration in Y-axis direction)²+(Acceleration in Z-axis direction)²)^{1/2}. In the present exemplary embodiment, the calculation unit 505 calculates the maximum value of the sum of squares of the physical quantities in the X-axis, the Y-axis, and the Z-axis directions as the impact value.

The calculation unit 505 performs the above-described calculation when the impact is detected by the impact detection unit 502.

The impact magnitude determination unit 506 determines the impact magnitude at the impact position based on the impact value obtained through calculations by the calculation unit 505 and the impact position determined by the impact position determination unit 504. In this case, the impact magnitude determination unit 506 may determine the impact value acquired by the calculation unit 505 simply as the impact magnitude at the relevant impact position. However, in the present exemplary embodiment, the impact magnitude determination unit 506 determines the impact magnitude at the impact position in consideration of the impact position on the radiation imaging apparatus 110. In this case, for example, in consideration of the distance between the impact detection sensor 1111 and the impact position determined by the impact position determination unit 504, the impact magnitude determination unit 506 calculates a value based on the distance from the impact value acquired by the calculation unit 505 to determine the impact magnitude at the impact position.

The memory unit 507 constitutes a storage unit for storing information about the impact position acquired as a result of the determination by the impact position determination unit 504 and information about the impact magnitude acquired as a result of the determination by the impact magnitude determination unit 506. The memory unit 507 stores other information required for the present invention.

The control unit 508 performs control to change subsequent operations in the radiation imaging system 100 according to the impact position information and the impact magnitude information stored in the memory unit 507. For example, the control unit 508 communicates with the information processing apparatus 130 via the communication unit 117 and then performs control to change the contents to be displayed on a display unit of the information processing apparatus 130.

For example, based on the impact position information stored in the memory unit 507, the control unit 508 controls operation diagnosis on each construction unit stored in the housing 110a of the radiation imaging apparatus 110. In the case of this configuration, for example, the information processing apparatus 130 acquires the impact position information and the information about the type of the radiation imaging apparatus 110 from the radiation imaging apparatus 110, and, based on these pieces of information, determines procedures of operation diagnosis to be conducted on each construction unit stored in the housing 110a of the radiation imaging apparatus 110. The information processing apparatus 130 which makes this determination includes a determination unit. The control unit 508 controls operation diagnosis on each construction unit stored in the housing 110a of the radiation imaging apparatus 110, based on the determination by the information processing apparatus 130.

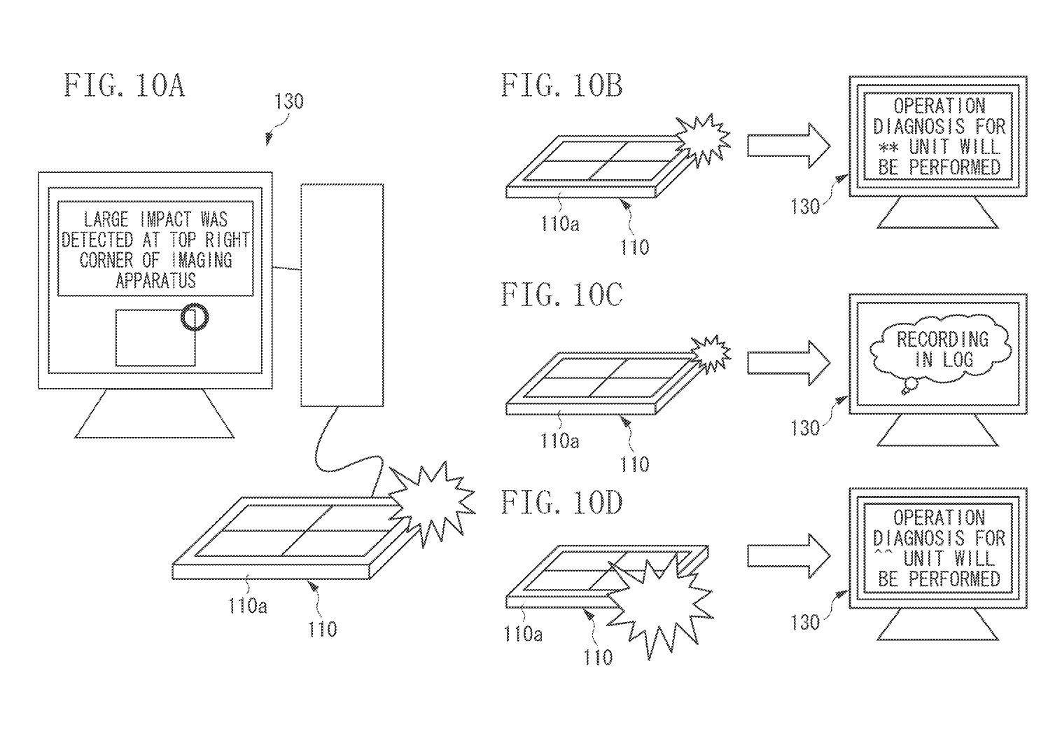

The control unit 508 performs other control related to the processing required for the present invention. Specific examples of the control processing by the control unit 508 will be described below with reference to FIGS. 10A to 10D.

FIGS. 10A to 10D illustrate examples of control processing by the control unit 508 illustrated in FIG. 5.

FIG. 10A illustrates a case where the impact magnitude acquired as a result of the determination by the impact magnitude determination unit 506 is equal to or greater than a predetermined threshold value, and the control unit 508 displays the relevant information together with the impact position on the display unit of the information processing apparatus 130 to visually notify a user of the impact in an easily understood manner.

FIG. 10C illustrates a case where the impact magnitude acquired as a result of the determination by the impact magnitude determination unit 506 is smaller than a predetermined threshold value, and the control unit 508 does not perform operation diagnosis on a structural member related to the impact position but performs only recording on a log.

FIGS. 10B and 10D illustrate a case where, for example, the impact magnitude acquired as a result of the determination by the impact magnitude determination unit 506 is equal to or greater than a predetermined threshold value, the control unit 508 performs, based on a relation between the impact position and the structural member relevant to the impact position, operation diagnosis on the relevant structural member. By actively performing self-diagnosis in this way, downtime can be reduced.

Specific examples of control processing by the control unit 508 illustrated in FIG. 5 will be described below.

Depending on the impact position described above with reference to FIGS. 7 and 8, the control unit 508 changes the execution order of operation diagnosis (performs operation diagnosis sequentially from a structural member determined to possibly be defective) or changes structural members to be subjected to operation diagnosis (performs operation diagnosis only on the structural member determined to be possibly defective).

For example, the control unit 508 determines the standard execution order of operation diagnosis as follows:
<1> Battery unit check
<2> Sensor drive check (the radiation detection unit 113, the drive IC 114, the amplifier IC 115, and the ADC 116)
<3> Digital image data forming unit check (the radiation detection unit 113, the drive IC 114, the amplifier IC 115, and the ADC 116)
<4> (Wireless) communication unit check
<5> Memory unit check
<6> Power button check
<7> Infrared communication unit check
<8> LED unit check

For example, when the impact position is the position of the top surface [1] illustrated in FIG. 7, the control unit 508 performs operation diagnosis giving priority to “<2> Sensor drive check.”

When the impact position is the position of the top right corner [2] illustrated in FIG. 7, the control unit 508 performs operation diagnosis giving priority to “<3> Digital image data forming unit check.”

When the impact position is the position of the right-hand side surface [3] illustrated in FIG. 7, the control unit 508 performs operation diagnosis giving priority to “<4> (Wireless) communication unit check.”

When the impact position is the position of the bottom right corner [4] illustrated in FIG. 7, the control unit 508 performs operation diagnosis giving priority to “<3> Digital image data forming unit check.”

When the impact position is the position of the bottom surface [5] illustrated in FIG. 7, the control unit 508 performs operation diagnosis not on a priority basis but, for example, in the above-described standard order.

When the impact position is the position of the bottom left corner [6] illustrated in FIG. 7, the control unit 508 performs operation diagnosis giving priority to “<3> Digital image data forming unit check.”

When the impact position is the position of the left-hand side surface [7] illustrated in FIG. 7, the control unit 508 performs operation diagnosis giving priority to “<3> Digital image data forming unit check.”

When the impact position is the position of the top left corner [8] illustrated in FIG. 7, the control unit 508 performs operation diagnosis giving priority to “<3> Digital image data forming unit check.”

When the impact position is the position of either the front or rear surface of the radiation imaging apparatus 110, the control unit 508 performs operation diagnosis not on a priority basis but, for example, in the above-described standard order.

As described above, the control unit 508 performs control to change subsequent operations in the radiation imaging system 100 by analyzing the impact position and the impact magnitude.

If a failure occurs in the radiation imaging apparatus 110 due to the impact, a causal defective portion can be determined by acquiring information about the relevant impact position.

In consideration of this point, the present exemplary embodiment is also applicable to the following mode.

For example, the memory unit 507 prestores a database for associating impact positions with defective portions. The memory unit 507 storing this database constitutes a storage unit.

For example, if the failure occurs in the radiation imaging apparatus 110, the control unit 508 identifies a defective portion based on the above-described database stored in the memory unit 507 and the impact position determined by the impact position determination unit 504. The control unit 508 which performs processing for identifying the relevant defective portion constitutes an identification unit.

If the identified defective portion is the battery unit 202, the control unit 508 transmits a message “Replace Battery” to the information processing apparatus 130 and displays the relevant information for prompting the user to replace the battery unit 202. Alternatively, the control unit 508 may transmit a message “Connect Sensor Cable to Connector Contact Portion, and Operate on Power Supplied from Connector Contact Portion” to the information processing apparatus 130 to display the relevant information.

When the identified defective portion is the wireless communication unit, the control unit 508 transmits a message “Use Wired communication Instead of Wireless Communication” to the information processing apparatus 130 and displays a warning not to use wireless communication. Alternatively, if a memory for recording image data is detachable, the control unit 508 transmits a message “Detach Memory before Imaging” to the information processing apparatus 130 and displays the relevant information.

The present exemplary embodiment determines the impact position and the like and therefore enables figuring-out what kind of impact has been applied to the radiation imaging apparatus 110 when it receives the impact. This makes it easy to identify a defective portion. As a result, even if the radiation imaging apparatus 110 becomes defective by an impact, it becomes possible to quickly recover the radiation imaging apparatus 110 and reduce downtime. Further, by determining the impact position, the possibility of incorrect detection can be reduced in the automatic detection mode.

A second exemplary embodiment of the present invention will be described below.

While, in the first exemplary embodiment, an acceleration sensor is used as the impact detection sensor 1111, the present invention is not limited thereto.

For example, in the second exemplary embodiment, an angular velocity sensor is used as the impact detection sensor 1111. In this case, since the impact detection sensor 1111 detects the orientation of the radiation imaging apparatus 110 when dropped, it becomes possible to identify the portion which has been turned downward and fallen, and determine the impact position.

Further, for example, the second exemplary embodiment is also applicable to a mode in which the above-described angular velocity sensor and the acceleration sensor according to the first exemplary embodiment are used together as the impact detection sensor 1111. In this case, the impact magnitude can also be determined.

Furthermore, for example, the second exemplary embodiment is also applicable to a mode in which a contact sensor and the acceleration sensor according to the first exemplary embodiment are used together as the impact detection sensor 1111. In this case, it becomes possible to determine the contact position, i.e., the impact position based on information from the contact sensor, and to determine the impact magnitude based on information from the acceleration sensor.

A third exemplary embodiment of the present invention will be described below.

In the above-described first exemplary embodiment, “Incorrect Detection” may arise when an impact is applied to the radiation imaging apparatus 110 in the “Automatic Detection Mode.” More specifically, when a fluctuating image signal exceeds a detection threshold value, “Incorrect Detection” of radiation may occur even if radiation which penetrates the subject H is not actually input.

In the first exemplary embodiment, it is the impact position that is determined. Therefore, except in a case where the subject H is placed on the radiation imaging apparatus 110 (for example, in a case where the radiation imaging apparatus 110 takes the impact from a side surface), the overall control circuit unit 111 determines that the apparatus has taken the impact even if the image signal exceeds the detection threshold value. In this case, the overall control circuit unit 111 performs control to stop electric charge accumulation in the photoelectric conversion element 311 for each pixel 310 of the radiation detection unit 113.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-120771, filed Jun. 11, 2014, which is hereby incorporated by reference herein in its entirety.

What is claimed is:

1. A radiation imaging apparatus for capturing a radiation image based on radiation which penetrates a subject, the radiation imaging apparatus comprising:
a detection unit configured to detect a physical quantity which is changed when the radiation imaging apparatus takes an impact; and
a position determination unit configured to determine an impact position subjected to the impact, based on the physical quantity detected by the detection unit.

2. The radiation imaging apparatus according to claim 1, wherein the detection unit detects the physical quantity in a plurality of axis directions in the radiation imaging apparatus, the radiation imaging apparatus further comprising a processing unit configured to perform determination processing for determining an output waveform type with respect to the physical quantity in each axis direction of the plurality of axis directions, and
wherein the position determination unit determines the impact position according to the type of the output waveform in each axis direction obtained as a result of the determination processing by the processing unit.

3. The radiation imaging apparatus according to claim 2, wherein the processing unit performs determination processing for determining whether an output waveform type is a first output waveform, a second output waveform, or a third output waveform,
wherein the first output waveform has a time period equal to or longer than a predetermined period, wherein, during the first output waveform time period, a magnitude is equal to or greater than a positive first threshold value,
wherein the second output waveform has a time period equal to or longer than a predetermined period, wherein, during the second output waveform time period, a magnitude is equal to or smaller than a negative second threshold value, and
wherein the third output waveform is other than the first and the second waveforms.

4. The radiation imaging apparatus according to claim 2, wherein the detection unit further detects the impact when a magnitude of the detected physical quantity is equal to or greater than a predetermined threshold value, and
wherein, when the impact is detected by the detection unit, the processing unit performs the determination processing.

5. The radiation imaging apparatus according to claim 4,
wherein the detection unit detects the physical quantity in a plurality of axis directions in the radiation imaging apparatus, and
wherein the calculation unit calculates the impact value based on a sum of squares of the physical quantities in a plurality of the axis directions.

6. The radiation imaging apparatus according to claim 4,
wherein, when a magnitude of the detected physical quantity is equal to or greater than a predetermined threshold value, the detection unit further detects the impact, and
wherein the calculation unit performs the calculation when the impact is detected by the detection unit.

7. The radiation imaging apparatus according to claim 4, further comprising:
a storage unit configured to store information about the impact position acquired as a result of the determination by the position determination unit, and to store information about the impact magnitude acquired as a result of the determination by the magnitude determination unit; and
a control unit configured to perform control to change subsequent operations according to the information about the impact position and the information about the impact magnitude stored in the storage unit.

8. The radiation imaging apparatus according to claim 1, further comprising:
a calculation unit configured to calculate an impact value based on the physical quantity detected by the detection unit; and
a magnitude determination unit configured to determine a magnitude of the impact at the impact position determined by the position determination unit, based on the impact value acquired through calculations by the calculation unit and the impact position determined by the position determination unit.

9. The radiation imaging apparatus according to claim 1, further comprising a power supply unit configured to supply power to the detection unit, even when power is not input to the relevant radiation imaging apparatus.

10. The radiation imaging apparatus according to claim 1, further comprising:
a storage unit configured to store a database by associating impact positions with defective portions; and
an identification unit configured to identify, when the radiation imaging apparatus becomes defective, a defective portion based on the database and the impact position determined by the position determination unit.

11. The radiation imaging apparatus according to claim 1, further comprising:
a radiation imaging unit configured to detect the radiation which penetrates the subject to acquire an image signal;
a storage unit configured to store the radiation image obtained based on the image signal; and
a housing configured to store the radiation imaging unit and the storage unit,
wherein the position determination unit determines, as the impact position, a position subjected to the impact from external.

12. The radiation imaging apparatus according to claim 11, wherein the housing has at least four side surfaces and a plurality of corner portions, wherein each corner portion is formed of two adjacent side surfaces out of the at least four side surfaces, and
wherein the position determination unit determines which corner portion of the plurality of corner portions was subjected to the impact.

13. The radiation imaging apparatus according to claim 11, wherein the housing has an approximately rectangular parallelepiped shape, and
wherein the position determination unit determines which of four corner portions of the housing has been subjected to the impact.

14. The radiation imaging apparatus according to claim 1, further comprising:
a radiation imaging unit configured to detect the radiation which penetrates the subject to acquire an image signal;
a drive circuit unit configured to apply a drive signal to the radiation imaging unit;
an analog-to-digital (A/D) conversion unit configured to convert the image signal into digital data;
a storage unit configured to store the radiation image obtained based on the image signal;
a battery unit configured to supply power to the radiation imaging unit;
a power source control circuit unit configured to convert power from the battery unit into a predetermined voltage, and supply the predetermined voltage;
a wireless communication unit configured to wirelessly communicate with an external apparatus;
a control unit configured to control operations by the radiation imaging apparatus; and
a housing configured to house the radiation imaging unit, the drive circuit unit, the A/D conversion unit, the storage unit, the battery unit, the power source control circuit unit, the wireless communication unit, and the control unit,
wherein, based on the determined impact position, the control unit controls operation diagnosis on each unit provided in the housing.

15. The radiation imaging apparatus according to claim 1,
wherein the detection unit has an acceleration sensor, and
wherein the acceleration sensor detects a value indicating acceleration as the physical quantity.

16. A radiation imaging system comprising:
a radiation imaging apparatus according to claim 15; and
an information processing apparatus configured to communicate with the radiation imaging apparatus,
wherein the information processing apparatus includes a determination unit configured to determine procedures of operation diagnosis on each unit provided in a housing, based on information about the impact position and information about a type of the radiation imaging apparatus, and
wherein the radiation imaging apparatus controls the operation diagnosis based on the determination by the determination unit.

17. A radiation imaging system comprising:
the radiation imaging apparatus according to claim 1; and
an information processing apparatus configured to communicate with the radiation imaging apparatus.

18. A method for controlling a radiation imaging apparatus for capturing a radiation image based on radiation which penetrates a subject, the method comprising:
detecting a physical quantity which is changed when the radiation imaging apparatus takes an impact; and
determining an impact position subjected to the impact, based on the detected physical quantity.

19. The method for controlling a radiation imaging apparatus according to claim 18, the method further comprising:
calculating an impact value based on the detected physical quantity; and
determining a magnitude of the impact at the determined impact position, based on the impact value acquired through calculations and the determined impact position.

20. A non-transitory computer-readable storage medium storing a program to cause a radiation imaging apparatus, for capturing a radiation image based on radiation which penetrates a subject, to perform a method, t, the method comprising:
detecting a physical quantity which is changed when the radiation imaging apparatus takes an impact; and
determining an impact position subjected to the impact, based on the detected physical quantity.
